(19) United States
(12) Patent Application Publication
(10) Pub. No.: US 2002/0161345 A1
(43) Pub. Date: Oct. 31, 2002

(54) Wound splashguard

(76) Inventor: Matthew McMillin, Farmington, IL (US)

Correspondence Address: BARNES & THORNBURG, 2600 CHASE PLAZA, 10 LASALLE STREET, CHICAGO, IL, 60603

(21) Appl. No.: 09/844,099

(22) Filed: Apr. 27, 2001

(51) Int. Cl. A61M 35/00; A61F 13/00

(52) U.S. Cl. 604/304; 604/290

(57) ABSTRACT

The present invention is directed to a wound splashguard which includes a wound cover for blocking fluid splatter, and an adjustable fluid source mount for allowing movement of a fluid source from a first position to at least a second position to re-direct a fluid spray relative to the wound while the splash guard is maintained in a substantially stationary position. The invention is further directed to a method of using the adjustable mount which allows the practitioner to move the fluid source so that the fluid spray can irrigate a larger portion of a wound without having to move the splashguard thereby increasing irrigation efficiency and reducing the risks of needle stick injury.

### FIELD OF THE INVENTION

This invention relates to a medical device used during medical procedures such as wound cleansing and irrigation. In particular, the invention relates to a handheld wound splashguard which prevents the patient's body fluids from contaminating medical personnel during these procedures.

### BACKGROUND OF THE INVENTION

Wounds such as lacerations, abrasions, puncture wounds, and deep cuts are often contaminated with particulate debris, including dirt or grass, as well as fragments of glass, metal or rock. These contaminates may harbor high levels of bacteria and other harmful microorganisms, and to prevent sepsis and infection, these wounds should be cleansed of the debris as a step in the treatment regime for the wound.

In an clinic or emergency room setting, cleansing a patient's wounds is often accomplished by washing the wounds with a sterile/antiseptic irrigation fluid. The most common method of washing these wounds is to use a syringe containing the fluid or mechanical pump with a fluid reservoir attached to a hypodermic needle. Sufficient fluid is then injected through the needle and passed over the wound's surface to wash away the debris. Depending on the size of the wound and the amount of contamination in the wound, the amount of irrigation fluid used during wound irrigation can vary from about less than one hundred milliliters to several liters.

The method can involve inserting a hypodermic syringe needle into the wound cavity, and using the syringe to force irrigation fluid across the wound's surface, thereby forcing out the debris or contaminated material. In addition, some large wounds, particularly abrasions, can require additional irrigation along multiple sites across the length of the wound. Additional irrigation can be accomplished by refilling/replacing the syringe and reusing the needle, or by replacing of both the syringe and needle.

This irrigation procedure is not without its risks. First, upon contact with the wound, the irrigation fluid becomes contaminated with the patients blood and lymph. This contaminated fluid can then be splattered from the wound and onto adjacent medical personnel. The fluid can travel a significant distance from the wound, and depending in part on the pressure being used to irrigate the wound, the fluid can contaminate medical personnel. When the patient has a concurrent infectious disease, such as hepatitis C, hepatitis B or AIDS, this contaminated fluid can be of particular concern to the medical personnel.

Also, the hypodermic needle used during the irrigation can also become contaminated by the patient's blood. When more than one syringe full of fluid is necessary to cleanse the wound, or if the syringe needs to be moved to a different site along the wound's surface, the removal and replacement of the needle represents a needle stick hazard. Since the needle is contaminated with the patient's blood, a needle stick is potentially a greater risk to medical personnel in terms of infectious disease transmission. In addition, the contaminated needles are typically disposed of in SHARPS containers. SHARPS containers are medical waste containers specifically designed for the disposal of this type of medical waste. Once the containers are filled, they are specially sealed and removed from the hospital. However, the removal and disposal of these containers is both hazardous and relatively expensive. Thus, there is a need during wound irrigation to reduce the chance of needle stick injuries and the number of contaminated hypodermic needles produced during medical procedures.

At present, medical personnel are taking several measures to reduce the aforementioned risks of transmission of disease from patient to physician. These measures include the use of protective face shields and the use of hand-held wound guards. Face shields are clear plastic barriers attached to head pieces, and are worn by practitioners to prevent backsplash contamination. Although a face shield prevents contaminated fluid from reaching the practitioners face, the shields are cumbersome in use, can be slightly off-putting to patients, and offer no protection to the rest of the practitioners body or adjacent personnel. Nor do the shields address the aforementioned needle stick problems associated with the wound irrigation.

Prior wound guards provide a barrier between medical personnel and the contaminated fluid. However, the currently available wound guards are less efficient to use than is desirable. To use such wound guards, the practitioner typically must hold the wound guard with one hand while inserting the needle of the irrigation syringe through an aperture or mount in the guard, or through the guard itself. When the syringe is empty, it must be removed, refilled and replaced. Replacing the syringe typically requires the practitioner to reinsert the syringe through the wound guard which brings the hypodermic needle into close proximity to the practitioner's hand holding the wound guard. This proximity creates an additional risk of a needle stick injury for the practitioner. Frequently, particularly when treating large or elongated wounds, the practitioner must move the syringe to repeatedly irrigate a different portion of the wound. In such cases, the practitioner either leaves the syringe in the wound guard, and moves both guard and syringe at once, or the physician removes the syringe from the guard, moves the guard, and replaces the syringe. Moving both the syringe and guard at once requires the practitioner to use both hands, which can be relatively cumbersome and inefficient in some instances and may involve additional risk of needle stick injury. Removing and replacing the syringe, while less cumbersome, can create a higher risk of needle stick injury.

Finally, the production and storage costs associated with typical disposable wound guards can be higher than is desired. Typical guards incorporate multiple parts, which increases production costs. Also, some guards cannot be easily stacked for efficient shipping and storage. Therefore, a need exists for a wound splash guard that can irrigate a larger portion of a wound or abrasion without being moved. There is also a need for a wound splash guard that can retain a hypodermic needle and thereby minimize needle stick injury due to the necessity of repeatedly inserting the syringe into the guard. There is also a need for a wound guard that can be economically manufactured and efficiently shipped and stored.

### SUMMARY OF THE INVENTION

The present invention is directed to a wound splashguard which includes a wound cover for blocking fluid splatter, and an adjustable fluid source mount for allowing movement of the fluid source from a first position to other positions to redirect the fluid spray relative to the wound while the splash guard is maintained in a substantially stationary position. The adjustable mount allows the practitioner to move the fluid source so that the fluid spray can irrigate a larger portion of a wound without having to move the splashguard, thereby increasing efficiency and reducing the risks of needle stick injury. Preferably, the mount of the wound guard includes a cup member which snugly retains a fluid source of a predetermined dimension. Use of such a cup member can ease the movement of the splashguard from position to position and further reduce the risk of needle stick injuries when a syringe is used as a fluid source.

In one embodiment of the invention, the wound cover forms a dome for covering the wound, and the adjustable fluid source mount is located on the surface of the wound cover. The adjustable fluid source mount preferably includes a corrugated tube for moving the fluid source from a first position to at least a second position and thereby re-directing the fluid spray around the wound. The preferred fluid source mount includes a cup member which is dimensioned to receive and retain by friction fit the needle support portion or base of a syringe. The cup defining at least a portion of the passageway extending through the wound cover for receiving a needle attached to the syringe. In this preferred embodiment, the wound cover and adjustable fluid source mount are integrally formed from a generally transparent plastic material and include a conical section. The wound cover also includes at least one drainage aperture to channel the flow of blood and irrigation fluid away from the wound site.

In another embodiment of the invention, the splashguard includes a wound cover and an adjustable fluid source mount having a ball-in-socket arrangement. Preferably, the socket is integrally formed in the wound cover and the ball member is rotatably mounted in the socket. The ball member includes a cup member having a cup aperture for retaining the fluid source and a passageway communicating with the interior of the splashguard. Other alternative embodiments of the invention are contemplated in which the adjustable fluid source mount may include a pivot member and hinge, a sliding member in slot arrangement or flexible tube.

### BRIEF DESCRIPTION OF THE DRAWINGS

FIG. 1 depicts a perspective view of one embodiment of the wound splashguard of the invention shown with a syringe and needle inserted into the splashguard.

FIG. 2 depicts a side view of the splashguard of FIG. 1.

FIG. 3 depicts a plan view of the splashguard of FIG. 1.

FIG. 4 depicts an end view of the splashguard of FIG. 1 illustrating the bending of the adjustable fluid source mount in phantom.

FIG. 5 is an enlarged cross-sectional view taken along lines 5—5 of FIG. 2.

FIG. 6 is an alternate embodiment of the splashguard invention in cross-section.

FIG. 7 is a perspective view of another alternate embodiment of the splashguard of the invention.

FIG. 8 is a fragmentary plan view of the splashguard.

FIG. 9 is a cross-sectional view taken along lines 9—9 of FIG. 8.

FIG. 10 is a cross-sectional view taken along lines 10—10 of FIG. 9.

### DETAILED DESCRIPTION OF THE INVENTION

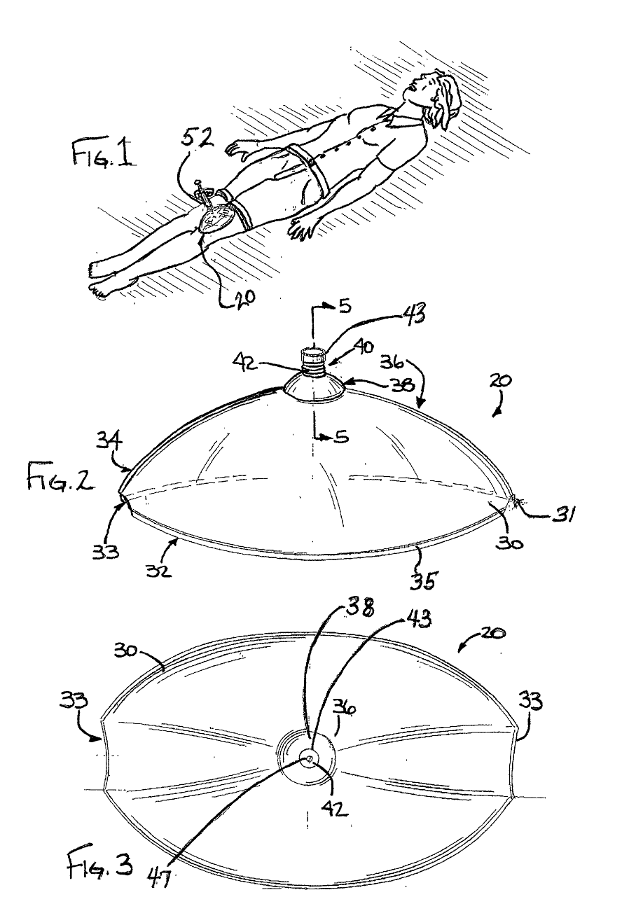

The present invention relates to a wound splashguard for use during wound irrigation procedures. FIGS. 1-5 generally depict a wound splashguard 20 in accordance with one embodiment of the invention. The splashguard 20 generally includes a wound cover 30 and an adjustable fluid source mount 40 (hereinafter “the mount 40”), which includes a corrugated tube 42 and cup member 43. As can be best seen in FIG. 1, the mount 40 is preferably integrally formed with the wound cover 30. It is contemplated that the splashguard 20 may be fashioned from more than one piece. For example, the adjustable mount could be adhered to or snap fit onto the wound cover.

The wound splashguard 20 is preferably made from a generally transparent, plastic resin so that the wound and hypodermic needle can be clearly visualized through the wound cover 30. The splashguard of the invention be made from a variety of plastic resins commonly used in disposable surgical devices by conventional injection molding or blow molding techniques. For example, the wound cover can be formed from transparent materials such as polyvinyl chloride or polypropylene injected into a suitable mold. Although in the embodiment depicted in FIGS. 1-5 it is preferred that the splashguard be semi-rigid and hemispherical in shape, it is further contemplated that the splashguard of the invention may range from flexible to substantially rigid, and from substantially transparent to slightly opaque, depending on the materials selected for the splashguard's manufacture.

For the purpose of this description, the exterior surface of the splashguard 20 is defined as the surface which faces the practitioner during the application and use of the wound cover, and the interior surface of the splashguard 20 is defined as the surface of the wound cover which faces the patient. The space between the interior surface of the splashguard and the patient is defined as the “interior shield space.”

The splashguard 20 of FIG. 1 and FIG. 2, includes a wound cover 30 for blocking the splatter or backsplash of irrigation fluid which has a generally oval shaped base 32 (FIG. 2), from which extends a generally transparent dome 36. The generally oval shaped base 32 and dome 36 depicted are configured to provide greater coverage for elongated laceration wounds and cuts. It is contemplated that the wound cover may be configured with different shapes, as long as the shapes provide a barrier to prevent splatter and can be readily held by the practitioner. For example, but without limitation, a generally round base and hemispherical dome may also be employed to provide a more efficient barrier for irrigating deep puncture wounds, or a generally polygonal base and rounded box shaped dome may be employed for general wound irrigation.

As best seen in FIG. 1, the base 32 has two raised areas located along the elongated axis of the perimeter which define a pair of drainage apertures 31, 33 (FIG. 2). These drainage apertures are shaped to channel irrigation fluid and blood to flow away from the wound, but are sized to prevent or reduce the amount of fluid splatter through these apertures 31, 33. Also, a rim 35 of thickened transparent plastic for improving the dome's rigidity is located at the periphery of the base 32 of the generally transparent dome 36.

As seen best in FIG. 1, wound cover 30 has a generally frusto-conical portion 38 located at the approximate apex of the generally transparent dome 36. As depicted in this embodiment, this frusto-conical portion 38 is integrally formed with the transparent dome 36 of the wound cover 30. The frusto-conical portion 38 raises mount 40 above the exterior surface of the transparent dome 36, and encompasses a corresponding frusto-conical, interior space defined by the interior surface of the frusto-conical portion 38 of the dome 36. When several wound splashguards are stacked on top of each other for storage or packaging, the fluid source mount 40 fits within the conical space, enabling the wound splashguards 20 to be more efficiently stacked. As can be seen in FIG. 5, the wound cover 30 has an aperture 37 which passes through the wound cover to receive at least a portion of the fluid source so that at least a portion of the fluid source projects into the interior shield space to provide fluid spray to the wound.

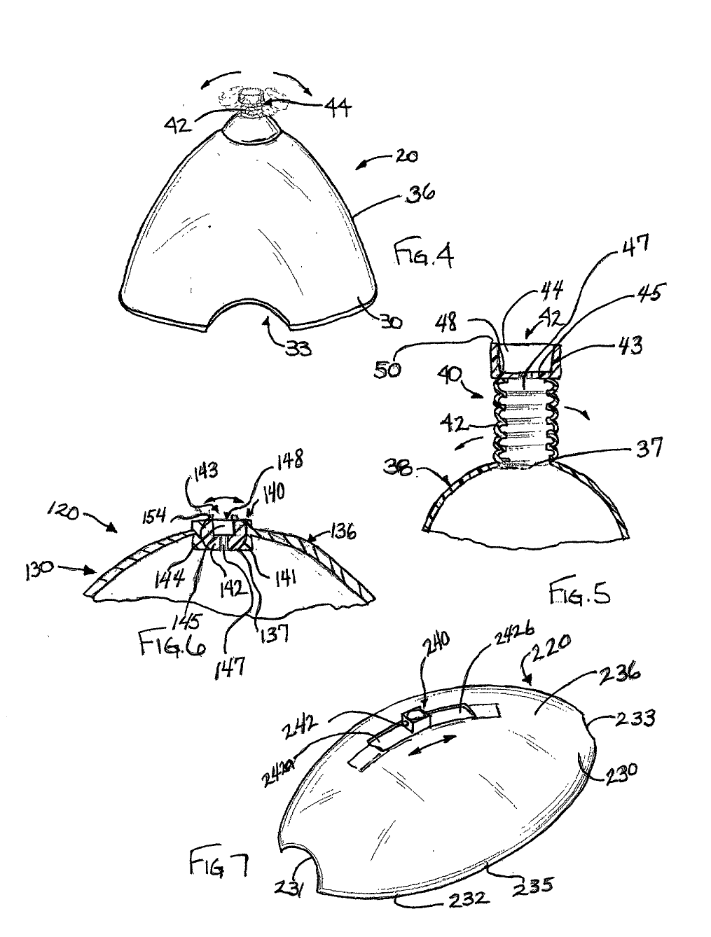

In the embodiment shown in FIGS. 1-5, the mount 40 is located on top of the conical section 38 and includes the corrugated plastic tube 42 and the cup member 43. The corrugated plastic tube is formed by alternating ridges and grooves of plastic material. The corrugated tube 42 is flexible so that the tube 42 can be bent by the practitioner to change the position of the fluid source to direct the fluid spray to different sections of the wound without substantial movement of the wound cover 30. Bending the corrugated tube 42 causes the ridges and grooves on the surface on the interior angle to collapse and the ridges and grooves on the exterior angle to expand as best illustrated in FIG. 4. It is contemplated that non-corrugated, flexible tubes may be used which are made of a resilient plastic material that bends laterally with pressure to allow the position of the fluid source to be altered to redirect the fluid spray. Of course, it is contemplated that the would splashguard of the present invention may be moved to more than one position if all portions of the wound cannot be reached by the fluid spray from a single position. Even in such a case, the splashguard of the invention will cut down considerably the number of times the splashguard must be moved when compared with conventional splashguards having a static mount.

The cup member 43 includes a cylindrical wall 44, stop surface 45, and passageway 47. The cylindrical wall 44 extends from the corrugated tube 42 and defines the periphery of cup aperture 48. Cup member 43 further includes a bottom wall or stop surface 45 which acts to prevent over-insertion of the fluid source into the passageway 47 and defines the bottom of the cup aperture. The passageway 47 extends through the cup member 43, tube 42, and opens into and is in fluid communication with the aperture 37 of the wound shield 30. The passageway 47 is dimensioned to receive and pass through to the interior of the shielded space the narrow portion of the fluid source which ejects the fluid spray (typically a needle). In the embodiment shown in FIGS. 1-5, cup member 43 is dimensioned and shaped to receive a standard cylindrical syringe with attached needle in cup aperture 48. It is contemplated that the cup member may be dimensioned and shaped to receive and retain fluid sources with a wide variety of shapes and sizes, including, for example, those having square, rectangular, pentagonal, hexagonal, or octagonal cross-sections.

The cylindrical wall 44 extends slightly laterally outwardly from stop surface 45 to upper edge 50 of cup 43 so that the preferred cup member aperture 48 of the cup member 43 has a slightly larger internal diameter at upper edge 50 than near the stop surface 45. As a result, the cup aperture is slightly tapered when viewed from upper edge 50 to stop surface 45 which provides a friction fit with an enlarged diameter portion of the syringe, typically, a needle support or reservoir portion, depending upon the syringe design. It is also contemplated that the cylindrical wall 44 may include an internal spiral flange which corresponds to a spiral ridge on the base of common LUER-LOCK needle support which is used to mount the needle of a syringe to a protective cover. By such friction fit, the needle and needle support of a fluid source may be retained by the splashguard 20 and it may be decoupled from the syringe or other fluid reservoir to refill the syringe. Then, the syringe can be recoupled with the retained needle portion so that the syringe may be refilled without having to re-insert the needle in the splashguard thereby reducing the risk of needle stick injury to the practitioner.

The fluid source may be any of the fluid sources provided to irrigate wounds in an emergency room or surgical suite. Typically, in an emergency room setting as shown in FIG. 1, a simple syringe 52 with a detachable needle is the fluid source. The needle has an 18 gauge or smaller opening to provide sufficient pressure to the fluid spray and typically has an exterior thread on an enlarged diameter needle support. Such exterior needle support threads are typically twisted onto an interior thread on the syringe to form a detachable coupling of needle support and syringe. In surgical suites, more elaborate fluid sources may be used which have a fluid pump or similar device to provide the pressurized irrigation spray to a needle or similar structure having an opening with a relatively small interior diameter.

Another embodiment of the invention is shown in FIG. 6 which includes a splashguard 120 having a wound cover 130 and adjustable fluid source mount 140 (hereinafter “mount 140”). The splashguard 120 is similar in most respects to that shown in FIGS. 1-5 with the exception of the mount 140. As shown in FIG. 6, the mount 140 includes a ball portion 142 which is received in a socket portion 141. As shown in FIG. 5, the socket 141 is integrally formed with the wound cover 130. The wound cover 130 and socket portion 141 are preferably integrally formed by injection molding or blow molding a plastic resin of the type discussed previously above. The socket portion 141 defines an aperture 137 formed in the wound cover 130.

The ball member 142 is preferably molded as a separate piece and has a cup member 143 defining a cup aperture 148 which receives a fluid source (not shown). The ball portion 142 is preferably dimensioned so that it can be snap-fit into the socket portion 141. The ball portion 142 is further designed and dimensioned to pivot and rotate within the socket so that the position of the fluid source can be altered to direct the fluid spray to different portions of the wound without movement of the shield 120. The ball 142 is preferably provided with an upper rim 154 which prevents the ball member 142 from over-pivoting or over-rotating such that the cup aperture 148 of the cup member 143 is obstructed by the socket 141.

The cup member 143 includes a cylindrical wall 144, stop surface 145, and passageway 147 similar to those described above for the embodiment of FIGS. 1-5. Cup aperture 143 is likewise tapered from near the upper rim 154 of the cylindrical wall 144 to the stop surface 145 to provide a friction fit with a portion of the fluid source of predetermined dimensions. As shown in FIG. 6, the splashguard 120 lacks a fructo-conical portion such as that shown in the embodiment of FIGS. 1-5. Such a structure may optionally be included, but is not preferred with the ball-in-socket adjustable mount since it is not deemed to be necessary to facilitate stacking as the ball-in-socket adjustment has a lower profile than the tube mount of FIGS. 1-5. The splashguard 120 is otherwise similar to the embodiment of FIGS. 1-5 having similar drainage base, apertures, rim, and dome 136 portions.

It should be noted that the change in position of the adjustable fluid source mount and fluid source, as shown for example in the embodiments of the invention of FIGS. 1-6, can also change the angle at which the fluid spray meets the wound. This can be advantageous when attempting to remove foreign material from a wound by irrigation. The design of prior splashguards typically directed the spray perpendicularly at the wound and made it difficult to alter the angle without either re-sticking the needle or moving the splashguard. It is further contemplated that the ball-in-socket arrangement of the mount 140 could be replaced with a pivoting cup (not shown) having pivot arms and receiving cavities formed in the wound cover. However, the ball-in-socket arrangement is preferred because it provides redirection of the fluid spray in multiple axis to accommodate wider wounds or abrasions with less movement of the splashguard.

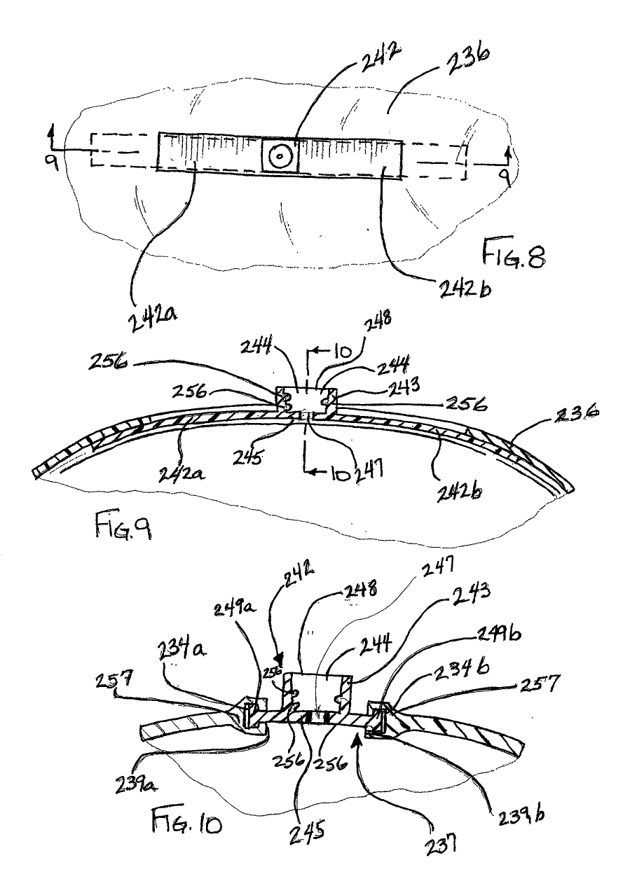

Another embodiment of the invention is shown in FIGS. 7-10, in which a splashguard 220 including a wound cover 230 and adjustable fluid source mount 240 (hereinafter “the mount 240”). The wound cover 230 is similar to those shown above in the embodiments of FIGS. 1-6, with the exception of some changes to accommodate the mount 240. The mount 240 includes a sliding member 242 having a cup member 243 which, similar to previously described embodiments, has a cylindrical wall 244, a stop surface 245 and passageway 247. As can be seen in FIG. 7, the sliding member 242 includes longitudinally extending arms 242a and 242b preferably made of a flexible resilient material. As shown in FIGS. 8-10, the wound cover 230 has slots or channels 234a and 234b positioned on the internal walls 239a and 239b defining aperture 237 which are dimensioned to receive guide members 249a and 249b located along the lateral edges of the sliding member 242. The guide members 249a and 249b are preferably continuous projections extending along both lateral edges of the sliding member from the longitudinal end of one arm 242a through the cup member 243 to the end of the other arm 242b. The guide members 249a and 249b retain the sliding member 242 within the slots 234a and 234b such that the cup member 243 and thereby fluid source may be slid along a substantial portion of the length of the splashguard 220 to direct the fluid spray to different portions of the wound without substantially moving the guard.

Depending on the thickness of the wound cover 230, a thickened ridge 257 adjacent to aperture 237 may be required to accommodate the slots 234 in the interior of the wound cover 230 as shown in FIG. 10. The guide members 249a and 249b of the sliding member 242 are preferably snap fit into channels 234a and 234b.

As can be best seen in FIGS. 9 and 10, the interior surface of the cylindrical wall 244 has a spiral groove 256 formed therein which may be engaged by a spiral flange on the exterior surface of a needle support (not shown) to securely retain the needle in the mount. The spiral flange on the needle support can be screwed down into the spiral groove 256 of the cup 243 to firmly secure the needle in the cup 243. The splashguard 220 is otherwise similar to the embodiment of FIGS. 1-5 above having a similar base 232, drain apertures 231 and 233, rim 235 and dome 236 portions.

To use the wound splashguard to assist in wound irrigation, the practitioner places the splashguard over the wound and inserts a fluid source containing irrigation fluid into the adjustable fluid source mount so that at least a portion of the fluid source passes through the aperture in the wound cover to the interior shielded space. Preferably, the practitioner pushes a portion of the fluid source, typically a needle support, downwardly into the tapered cup until it is retained by a friction fit. However, if a fluid source with an external spiral flange is selected, it may be screwed down into the cup member of the adjustable fluid source mount.

If the wound is large, elongated or the irrigation process would be facilitated by directing the fluid source from more than one angle, the practitioner can move the adjustable mount from a first position to at least a second position to re-direct the fluid spray along the wound without having to move the splashguard. Movement or adjustment of the position of the adjustable fluid source mount or fluid source as used herein means any change in position or orientation of the fluid source whether by sliding, pivoting, rotating or bending the mount which causes a significant redirection of the fluid spray. For example, this movement may be by the practitioner bending the tube 42 of the mount 40 of the embodiment of FIGS. 1-5, rotating or pivoting the ball member 142 of the embodiment of FIG. 6, or sliding the sliding member 242 of the embodiment of FIGS. 7-10.

If a refillable fluid source such as a syringe is used, the practitioner can refill or replace it with a full fluid reservoir. This can be accomplished by the practitioner decoupling the needle support portion of the fluid source (typically by unscrewing it from the fluid reservoir), refilling or replacing it with a full reservoir, and recoupling it to the retained needle support portion. As the fluid ejecting portion of the fluid source is preferably retained by the cup member of the splashguard, the practitioner can avoid re-sticking the splashguard when refilling or replenishing the fluid source. This feature can reduce the likelihood of needle stick injury to the practitioner.

After irrigation is complete, the wound splashguard is removed from the wound and the fluid source is disengaged from the splashguard. Preferably, in the emergency room setting, both the fluid source and splashguard are made of relatively inexpensive plastic resins and both may be disposed of by the practitioner. However, in the surgical suite setting, it may be desirable to have a reusable fluid source and splashguard that are made of a material which can withstand disinfection by autoclave or other suitable means. In this case, the practitioner removes the fluid source from the splashguard and places the wound guard in an appropriate location for disinfection with other surgical implements.

The applicant has provided description and figures which are intended as an illustration of certain embodiments of the invention, and are not intended to be construed as containing or implying limitation of the invention to those embodiments. It will be appreciated that, although applicant has described various aspects of the invention with respect to the specific embodiments, various alternatives and modifications will be apparent from the present disclosure which are within the spirit and scope of the present invention as set forth in the following claims.

What is claimed:

1. A splashguard for use in irrigating a wound with a fluid spray provided by a fluid source, comprising:
a wound cover for blocking fluid splatter during wound irrigation by the fluid spray, the wound cover having an aperture passing therethrough; and
an adjustable fluid source mount in communication with the aperture for receiving the fluid source and allowing movement of the fluid source from a first position to at least a second position to re-direct the fluid spray while the splashguard is maintained in a substantially stationary position.

2. The wound splashguard of claim 1, wherein the wound cover and flexible syringe mount are integrally formed from a generally transparent plastic material.

3. The wound splashguard of claim 1, wherein the splashguard is formed from a substantially transparent disposable plastic material.

4. The wound splashguard of claim 1, wherein the wound cover is a dome having an elongated base.

5. The wound splashguard of claim 1, further comprising at least one drain for fluid runoff.

6. The wound splashguard of claim 1, wherein the adjustable fluid source mount includes a flexible tube having a first end joined with the wound cover, and a second end of the tube extending to a cup member for receiving the fluid source, the flexible tubing defining an internal passageway extending from the second end of the tube to the aperture of the wound cover.

7. The would splashguard of claim 6 wherein the flexible tube includes a corrugated section with a plurality of ridges and grooves adapted to be flexed to allow movement of the fluid source from a first position to at least a second position to redirect the fluid spray.

8. The wound splashguard of claim 6, wherein the dome has a substantially hemispherical shape and a conical portion extending from the surface of the dome, the conical surface being integrally formed with the dome, and the adjustable fluid source mount extending from the surface of the conical surface.

9. The wound splashguard of claim 1, wherein the adjustable fluid source mount includes a cup member having a stop member and a wall portion extending from the perimeter of the stop portion, the stop portion being adapted to contact a portion of the fluid source to prevent over-insertion of the adjustable fluid source mount, the stop member and wall being arranged and dimensioned to define a fluid source aperture for receiving a fluid source.

10. The wound splashguard of claim 9, wherein the wall defining the fluid source aperture has opposing interior surfaces, the opposing interior surfaces tapering inwardly to less than a predetermined interior dimension such that an inserted fluid source having an exterior dimension greater than the predetermined interior dimension is retained in the fluid source aperture of the cap by friction fit.

11. The wound splashguard of claim 1, wherein the adjustable fluid source mount includes a ball member and a socket member located on the wound cover, the ball member being rotatably mounted in the socket and adapted to receive a fluid source whereby the fluid source may be moved from at least a first position to a second position to re-direct the fluid spray by rotation of the ball member while the splashguard is maintained in a substantially stationary position.

12. The would splashguard of claim 1 wherein the adjustable fluid source mount includes a cup member for receiving the fluid source, the cup member having a hinge portion for pivotally mounting to the wound cover whereby the fluid source may be moved from a first position to at least a second position to re-direct fluid spray by pivoting the cup member while the splashguard is maintained in a substantially stationary position.

13. The would splashguard of claim 1 wherein the adjustable fluid source mount includes a sliding member for receiving the fluid source, the sliding member having at least one guide member, and wherein the wound cover includes at least one slot for slidably retaining the at least one guide member whereby the fluid source may be moved from a first position to at least a second position to re-direct the fluid spray by sliding the slidable member along a length of the slot while the splashguard is maintained in a substantially stationary position.

14. The wound splashguard of claim 1 further comprising a raised porition along the perimeter of the wound cover defining a drainage aperture.

15. A wound splashguard for blocking fluid splatter, comprising:
a wound cover for blocking fluid splatter, the wound cover having an aperture passing therethrough, a generally oval shaped base and a generally hemispherical surface extending from the base; and
an adjustable fluid source mount in the fluid communication with the wound cover.

16. The wound splashguard of claim 15, wherein the generally hemispherical surface further includes a generally frusto-conical portion extending from the hemispherical surface, and the adjustable fluid source mount is located on the frustoconical portion.

17. The wound splashguard of claim 15, further comprising a drainage aperture positioned along the perimeter of the oval shaped base.

18. The wound splashguard of claim 15, wherein the adjustable fluid source mount includes a corrugated tube having a first end joined to the wound cover and a second end adapted to receive a fluid source, the corrugated tube defining a passageway extending from the second end through the wound cover.

19. The wound splashguard of claim 15, wherein the splashguard is integrally formed from a generally transparent disposable plastic material.

20. A method of irrigating a wound, comprising the steps of:
placing a wound splashguard having an adjustable fluid source mount over the wound;
inserting a fluid source containing irrigation fluid into the adjustable fluid source mount;
irrigating the wound with the fluid source maintained in a first position to direct a first spray of irrigation fluid to a first portion of the wound;
adjusting the adjustable fluid source mount to move the fluid source to a second position to re-direct the fluid spray; and
irrigating the wound with the fluid source mount in the second position to direct the fluid spray to a second portion of the wound.

21. The method of irrigation of claim 20, further comprising the steps of detachably coupling a needle portion of the fluid source to the fluid source mount, removing a substantially empty fluid reservoir from the fluid source and coupling a substantially full fluid reservoir to the needle.

22. The method of claim 20, further comprising the steps of removing the wound splashguard from the wound, and disposing of the needle and splashguard in the medical waste container.

23. The method of claim 20 wherein the step of adjusting the adjustable fluid source mount includes bending a flexible tube from a first position to a second position to re-direct the fluid spray.
